(19) United States
(12) Patent Application Publication
(10) Pub. No.: US 2016/0305863 A1
(43) Pub. Date: Oct. 20, 2016

(54) A SCREENING METHOD FOR RHEOLOGICAL PROPERTIES OF MILK GEL

(71) Applicant: CHR. HANSEN A/S, Hoersholm (DK)

(72) Inventors: METTE CANTOR, Birkeroed (DK); Vera POULSEN, Vaerloese (DK); Gunnar OEREGAARD, Vaerloese (DK); Jeorgos TRIHAAS, Oelsted (DK); Sandra WIND, Alleroed (DK); Patrick DERKX, Tikoeb (DK)

(73) Assignee: CHR. HANSEN A/S, Høersholm (DK)

(21) Appl. No.: 15/100,787

(22) PCT Filed: Dec. 9, 2014
(86) PCT No.: PCT/EP2014/076999

§ 371 Date: Jun. 1, 2016

(30) Foreign Application Priority Data
Dec. 10, 2013 (EP) 13196374.6

(51) Int. Cl. G01N 11/02; G01N 33/04

(52) U.S. Cl. CPC G01N 11/02; G01N 33/04

(57) ABSTRACT

The present invention relates to the field of dairy technology, in particular it relates to a method for assessing rheological properties of acidified milk gels (milk gels), including determination of shear stress, gel firmness and water-holding capacity. The method can be used to determine rheological properties of acidified milk, e.g. yoghurt and fresh cheese, in a fast and reliable way. The present invention also relates to a method of screening for microbial cultures resulting in fermented milk with desired rheological properties. By using an automated microtiter-plate pipetting station equipped with a pressure sensor inside the air displacement barrel of each pipette, it is possible to monitor real-time changes in pressure, when aspirating and dispensing milk gels, and then correlate the pressure versus time data obtained with milk gel rheological properties such as shear stress, gel firmness and water-holding capacity.

### FIELD OF THE INVENTION

The present invention relates to the field of dairy technology, in particular it relates to a method for assessing rheological properties of acidified milk gels (milk gels), including determination of shear stress, gel firmness and water-holding capacity.

The method can be used to determine rheological properties of acidified milk, e.g. yoghurt and fresh cheese, in a fast and reliable way. The present invention also relates to a method of screening for microbial cultures resulting in fermented milk with desired rheological properties.

### BACKGROUND OF THE INVENTION

Milk gels are a type of soft solid. Their networks are relatively dynamic systems that are prone to structural rearrangements. The physical properties of milk gels can be described using a model for casein interactions, which includes a balance between attractive and repulsive forces. Attractive forces might be e.g. hydrophobic attractions, casein cross-links contributed by calcium phosphate nanoclusters and covalent disulfide cross-links between caseins and denatured whey proteins. Repulsive forces might be e.g. electrostatic or charge repulsions, mostly negative at the start of fermentation.

Milk gels such as yoghurts are prepared by fermentation of milk with bacterial cultures consisting of a mixture of Streptococcus thermophilus and Lactobacillus delbrueckii subsp. bulgaricus. Bacterial fermentation converts lactose and other sugars into lactic acid, which reduces the pH of milk. During acidification of milk, the pH decreases from approximately 6.7 to ≦4.6, which leads to milk gel formation. The acidification process results in the formation of three-dimensional network consisting of clusters and chains of caseins. There are two major types: set and stirred yoghurt. Set yoghurt is formed in retail pots as lactic acid bacteria ferment lactose and other sugars into lactic acid giving a continuous gel structure in the consumer container. In stirred yoghurt, the acidified milk gel formed during incubation in large fermentation tanks is disrupted by stirring, and the stirred product is usually transferred to buffer tanks and coolers through pumps and tubes.

Food rheology is the study of the deformation and flow of food materials. Yoghurt can be classified as pseudo-plastic material (contains a yield stress that has to be exceeded for flow to be initiated) that can be either a viscoelastic fluid (stirred or drinking yoghurt) or a viscoelastic solid (set yoghurt). Viscoelastic indicates the material has some of the elastic properties of an ideal solid and some of the flow properties of an ideal (viscous) liquid (Lee and Lucey, 2010). A rheometer and/or a texture analyzer are typically used to assess rheological properties of yoghurt, such as complex modulus and shear stress. Large deformation rheological properties are important since most products are the stirred-type, where the initial gels are sheared and stirred. One type of large deformation test is a stress overshoot experiment or constant shear rate test. Sensory textural attributes are often correlated with the results from large deformation instrumental tests, e.g. shear stress is related to viscosity and complex modulus is related to gel firmness. The ability to gather data on rheological properties of acidified milk gives manufacturers of dairy products an important “product dimension”. Knowledge of rheological characteristics (flow of matter) is valuable in predicting pumpability and pourability, performance in a dipping or coating operation, or the ease with which it may be handled, processed, or used.

The physical attributes of yoghurts, including the lack of visual whey separation and perceived viscosity, are crucial aspects of the quality and overall sensory consumer acceptance of yoghurts. For these reasons, measurements of shear stress, gel firmness and whey separation are often an integrated part of the quality control applied in almost all areas where fermented milk products are produced/processed and delivered for further use.

Texture is an important quality factor for fermented milk products such as yoghurt, and consumer acceptance is often very closely linked to texture properties. Yoghurt texture varies with process parameters, and is affected in particular by the choice of bacteria culture. Texture can be described either by sensory analysis (performed by a panel of trained people) or by rheological methods providing information about flow behavior and viscous/elastic characters of the product. In a fermentation of milk with lactic acid bacteria (LAB), lactose and other sugars are converted to lactic acid leading to a reduction of pH followed by aggregation of the casein micelles and other milk proteins. Lowering the pH of milk to pH 4.6 leads to neutralization of casein particles (loss of charge), which subsequently leads to casein particle aggregation, ultimately leading to increased viscosity and gel formation. Gels can be characterized by e.g. complex modulus (correlating to the sensory descriptor gel firmness) and shear stress (correlating to the sensory descriptor mouth thickness). Due to structural changes in the protein network by the acidification of LAB as well the production of exopolysaccharides (EPS) by the LAB, the texture of different fermented milk products varies significantly.

Sensory evaluation of texture is often carried out as part of full sensory profiling providing information about odor, flavor, taste and texture of the yoghurts in one analysis. Important texture properties for yoghurt are Gel firmness, Mouth thickness and Ropiness.
Gel firmness: Evaluated visually by slowly placing a spoonful of yoghurt on an untouched yoghurt surface and evaluate how long it keeps the structure before flowing out—the longer the time the higher the Gel firmness.
Mouth thickness: Evaluated when eating the yoghurt at ‘normal-high’ eating rate—the longer it takes to swallow the yoghurt the higher the Mouth thickness.
Ropiness: Evaluated visually by pulling up a spoonful of yoghurt and evaluate how long the threads (ropes) are hanging on the spoon—the longer the ropes the higher the ropiness.

For set yoghurts, Gel firmness and Mouth thickness are the most important parameters, whereas Mouth thickness and Ropiness are the key parameters for stirred yoghurts. Generally, ropy yoghurt is creamier, smoother (less granular), thicker, and less firm. Creaminess is often associated with high ropiness. Sensory textural attributes are often correlated with the results from large deformation instrumental tests, for instance shear stress is related to viscosity and complex modulus is related to gel firmness. Water-holding capacity of the milk gel is an important quality parameter for set yoghurts and fresh cheese.

A common rheological technique for describing the rheological characteristics of fermented milk is viscometry, where the resistance to shear is measured. The measurements are carried out on a rheometer, on which the flow properties can be obtained over a wide range of shear rates giving a complete viscometric characterization. The shear stress (deformation produced by a force acting parallel to the surface of a body) is measured as a function of shear rate (the rate of change of strain) resulting in a flow curve. More information can be obtained from a double flow curve made by increasing the shear stress and subsequently decreasing it (reference is made to FIG. 1 in Mezger, 2006). The loop area between the up- and downwards flow curves is called hysteresis and describes the yoghurts' ability to regain structure after deformation. The Apparent viscosity can be calculated as shear stress divided by shear rate. For Newtonian liquids, such as water, shear stress and shear rate are simply proportional, and hence the viscosity is constant. However, many food products e.g. yoghurt are shear-thinning, meaning that an increasing shear rate gives a less than proportional increase in shear stress. Therefore, viscosity for these products must be presented as a number of apparent viscosities at specified shear rates.

In oscillation measurements the samples taken from the fermented milk product are subjected to oscillatory motions (a sinusoidal stress). The method is non-destructive, and is carried out at very small deformations. Thus information about the yoghurts' initial firmness before manipulation (for example in the mouth) is obtained. Measured parameters are the storage modulus G′ (representing the elastic properties) and the loss modulus G″ (representing the viscous properties). From these the complex modulus G*, which is a measure of total gel firmness, can be calculated.

Often fat and protein reduction in milk is associated with many textural and functional defects in fermented milk products. Some products such as yoghurt and cheese can show spontaneous syneresis, where a whey layer is formed on the surface of the product during fermentation. This whey layer may be absorbed during cooling. Some EPS or other polymer-producing microbial cultures are able to increase the water-holding capacity of milk gels by binding water/whey and thereby increasing the moisture content. This is an important function because fat and protein reduction results in lower moisture in the nonfat substance. Whey separation (wheying-off) is defined as the expulsion of whey from the network, which then becomes visible as surface whey. Wheying-off negatively affects consumer perception of yoghurt and thus yoghurt manufacturers use stabilizers (e.g. pectin, gelatin, starch) or increase the total solids content of milk, especially the protein content, to prevent wheying-off. Spontaneous syneresis, which is contraction of gel without the application of any external force (e.g. centrifugation), is the usual cause of whey separation. Spontaneous whey separation is related to an unstable network, which can be due to an increase in the rearrangements of the gel matrix or it can be induced by damage to the weak gel network (e.g. by vibration or cutting). Whey expulsion from fermented milk products, or syneresis, is measured through the use of centrifugation, drainage of whey through a screen or mesh or using a siphon method. The centrifugation method is a measure of the water-holding capacity as a result of a high external force, i.e. resistance of the gel to compaction.

Standard rheology measurements require relatively large samples of fermented milk to be tested. Although these methods of measurement are accurate and reproducible, they are highly demanding in respect of the time required per sample, technical skills and precision. The state of the art equipment for measuring viscosity takes about 20 minutes per sample, and common for all such equipment, whether it is a robot that carries out the process or semi-automatically or manually, is that only one sample at a time can be tested. These measurements are very time consuming, due to the washing and changing step between each sample, making it difficult to be used in a high throughput screen.

Pressure-monitoring during an automated pipetting process is carried out for various purposes in a number of pipetting applications, e.g., to discard irregular pipetting processes (WO2002073215; Hamilton Bonaduz AG) or to provide fully automatic control of the pipetting process (EP1614468; Hamilton Bonaduz AG).

Enzyme activity in a liquid, by measuring changes in viscosity over time in two or more samples is described in WO2011107472 (Novozymes).

There is a need for improved methods for determining shear stress, gel firmness and/or water-holding capacity of milk gels prepared in the presence of one or more bacterial strains and for screening the bacterial strains for their ability to contribute to shear stress, gel firmness and/or water-holding capacity. The invention presented here is taking advantage of using pipetting pressure curve measurements of milk gels, allowing fast determination of the rheological properties of the milk gels in small sample volumes, and thereby giving an indication of the potential of the bacterial strains to improve texture of milk gels.

### SUMMARY OF THE INVENTION

In the food industry, rheological characteristics of milk gels are as mentioned an important quality parameter. Often in fermentations of milk, lactic acid bacteria or other bacteria are added to milk to provide a product with desired taste and texture, and to extend the shelf life of milk. Screening of microorganisms providing particular and wanted texture of a product is time consuming and laborious due the drawbacks of the prior art methods for measuring viscosity. The present inventors found that the method according to the present invention was particularly useful for determining rheological characteristics of milk gels. In particular the method could be used also for determination of several rheological characteristics, including shear stress, gel firmness and water-holding capacity of milk gels.

In one aspect the present invention provides a method to determine at least one of shear stress, gel firmness and water-holding capacity of milk gels, said method comprising:
(i) Providing one or more samples, each comprising a sample container and a sample, where at least one sample is said milk gel;
(ii) Measurements of headspace pressure during aspiration and/or dispense of the samples of step (i) to obtain pressure versus time data; and
(iii) determining at least one of shear stress, gel firmness and water-holding capacity of said milk gel from the pressure versus time data of step (ii);
wherein said shear stress is determined in step (iii) by correlating the pressure versus time data with shear stress; said gel firmness is determined in step (iii) by correlating the minimum pressure on the aspiration curve obtained from a non-mixed or centrifuged sample with complex modulus and said water-holding capacity is determined in step (iii) by correlating the bend-time-point on the aspiration curve from a non-mixed or centrifuged sample with water-holding capacity.

The present invention makes it possible to select the best strains with desired texturing properties, including shear stress, gel firmness and water-holding capacity, in fermented milk by high throughput screening, e.g. in less than 60 seconds for 96 different samples measured simultaneously, when a 96-well micro container such as micro-titer plate is used. By using an automated microtiter-plate pipetting station equipped with a pressure sensor inside the air displacement barrel of each pipette (from Hamilton Robotics TADM system, described in EP2009449 and U.S. Pat. No. 6,938,504), it is possible to monitor real-time changes in pressure, when aspirating and dispensing milk gels, and then correlate the pressure versus time data obtained with milk gel rheological properties such as shear stress, gel firmness and water-holding capacity. Pressure monitoring is performed by TADM (total aspiration and dispense monitoring) tool, which is an optional installation in liquid handling robot mentioned above.

It has recently been stated (EP2009449; Hamilton Bonaduz AG), that the larger the surface tension and the viscosity of a liquid is, the lower is the pressure inside the pipette tip at the time the liquid starts flowing into the pipette tip and the larger is the absolute value of the slope of the time characteristics of pressure when the plunger moves without any flow of liquid taking place yet. As a further general rule, it was stated, that the larger the viscosity of a liquid is, the smaller is generally the slope of the time characteristics of pressure when the movement of the plunger has stopped, but the flow of liquid is still continuing. However, it was concluded, that all these general rules are of a more qualitative nature, and therefore, despite the fact that they seem quite intuitive, are difficult to evaluate quantitatively for calculating differences in process variables to be applied when liquids having differing physical characteristics are to be dosed using a pipetting device.

The method to determine at least one of shear stress, gel firmness and water-holding capacity of milk gels may be realized in different modes of operation. In one embodiment, the method of the present invention is for determining shear stress. In another embodiment, the method is for determining gel firmness. In a further embodiment, the method of the present invention is for determination of both shear stress and gel firmness. In another embodiment, the method of the present invention is for determination of water-holding capacity.

Hence, according to the particular sample and the viscosity characteristics which are desirable to quantify, the sample manipulation and the calculations are performed accordingly.

Usage of liquid handling robots enables a uniform treatment of the samples, and moreover makes it possible to work with low-volume samples in a fast way. The method of the present invention makes it possible to automatically process numerous samples, suitable for high-throughput screening of milk gels for several rheological properties: shear stress, gel firmness and water-holding capacity (wheying-off), in a fast, reliable and reproducible way.

Texture is extremely important, especially for low-fat and low-protein products, and it can be improved by using EPS-producing cultures, to compensate for the loss of fat and protein. In the development process of generating high-EPS producing cultures, the increases seen in viscosity are becoming incremental, highlighting the need for accurate measurements in order to differentiate and identify the best candidates. The method of the present invention can differentiate between milk gels such as yoghurts that are close in viscosity, and is also useful during preparation of cheese as described in WO2008153387 (Nizo Food Research B.V.).

The present invention is very useful when it comes to determine, predict or select which microorganisms, such as bacteria, or mixtures of such microorganisms are best for producing fermented milk products, such as a yoghurt or cheese, with desired qualities. In particular, microorganisms, such as bacteria, and mixtures thereof which lead to fermented milk with undesired properties, such as extreme thickness, non-uniform viscosity, etc., may be identified, based on the method of the present invention.

Hence, in a second aspect the present invention provides a method for the ranking of microorganisms according to their ability to contribute to shear stress, gel firmness and/or water-holding capacity of milk gels prepared in the presence of said microorganisms, said method comprising:
(i) providing two or more samples, each comprising a sample container and a sample, of a milk gel prepared in the presence of at least one microorganism;
(ii) measurements of headspace pressure during aspiration and/or dipense of the two or more samples of step (i) to obtain pressure versus time data; and
(iii) ranking the microorganisms according to the relative pressure versus time data of the milk gels prepared in the presence of the at least one microorganism,
wherein
shear stress is a) inversely proportional with pressure versus time data on the aspiration curve and b) directly proportional with pressure versus time on the dispense curve;
gel firmness is inversely proportional with the minimum pressure on the aspiration curve obtained from a non-mixed or centrifuged sample; and
water-holding capacity is inversely proportional with the bend-time-point of the aspiration curve obtained from a non-mixed or centrifuged sample.

In a third aspect the present invention provides an apparatus for determination of at least one of shear stress, gel firmness and water-holding capacity of milk gels, said apparatus comprising an automated pipette system fitted with a pressure sensor in the pipette headspace and a software control system scheduling the sample manipulation and recording of the pressure versus time measurements during step (ii) of the method.

Further objects and advantages of the present invention will appear from the following description, and claims.

### BRIEF DESCRIPTION OF THE FIGURES

FIG. 1: Dispense pressure measured over time [ms] of 5 commercial yoghurt cultures with different viscosities.

FIG. 2: Aspiration pressure measured over time [ms] of 5 commercial yoghurt cultures with different viscosities.

FIG. 3: Shear stress at 300 s⁻¹ measured on a rheometer plotted against dispense pressure monitored at 6 seconds, by pipetting.

FIG. 4: Shear stress at 300 s⁻¹ measured on a rheometer plotted against aspiration pressure monitored at 13 seconds, by pipetting.

FIG. 5: Aspiration curves for duplicate samples of single strain Lactobacilli, differing in shear stress.

FIG. 6: Aspiration curves for duplicate samples of 6 low fat yoghurts, differing in shear stress.

FIG. 7: Rate of pressure change during aspiration for duplicate samples of 6 low fat yoghurts, differing in shear stress.

FIG. 8: Dispense curves for duplicate samples of 6 low fat yoghurts, differing in shear stress.

FIG. 9: Rate of pressure change during dispense for duplicate samples of 6 low fat yoghurts, differing in shear stress.

FIG. 10: Predicted (Horizontal-axis) versus observed (Vertical-axis) values of Shear Stress by the regression model.

FIG. 11: Predicted (Horizontal-axis) versus observed (Vertical-axis) values of Complex modulus by the regression model.

FIG. 12: Correlation between aspiration pressure of milk gel samples prepared for screening purposes by fermenting milk directly in a micro-titer plate (1 ml per well) and shear stress values for the same microbial cultures that were used for milk fermentations in a larger scale (baby bottles, 200 ml). A. TADM aspiration curves of milk gel samples prepared by fermenting milk with 14 different Lactococcus strains directly in a micro-titer plate (1 ml) for 20 h at 30° C. and then stored at 4° C. for 1 day. A volume of 500 μl was aspirated. B. Correlation between aspiration pressure values at 1 s for the 14 samples from (A) and shear stress at 300 s⁻¹ measured on a rheometer using milk gel samples prepared with the same cultures in a 200 ml scale, where the samples were inoculated and then incubated for 12-15 h until they reach pH of 4.55.

FIG. 13: Screening Lactococcus strains in 96 well plate using TADM A. TADM aspiration and dispense curves for 96 samples in a micro-titer plate represented by two replicates. Milk gel samples were prepared by fermenting milk with up to 96 Lactococcus strains directly in a micro-titer plate (1 ml) for 20 h at 30° C. and then stored at 4° C. for 1 day. A volume of 500 μl was aspirated. B. Aspiration pressure values at 1 s and 1.2 s for each sample from both replicates were averaged, sorted according to the pressure values (lowest to highest) and plotted. Strain 1 (present twice on the plate as high-shear stress control) and Strain 2 from (A) appear on the lower scale of (B), while milk samples and strains that did not acidify milk appear on the higher pressure scale (only one representative sample is shown). The majority of strains that have acidified the milk but did not result in high-viscous milk gel were situated between approximately −2000 and −2500 Pa under current measurement conditions.

FIG. 14: Analysis of water-holding capacity of milk gels using TADM. A. Method concept. Correlation coefficient R was calculated based on measuring whey phase length in milk fermented by three different Streptococcus thermophilus strains, 16 replicates for each strain, and time points when TADM curves bend. B. TADM curves of milk gels obtained by fermenting milk with three different strains, and non-fermented milk was used as a control. A volume of 700 μl was aspirated.

FIG. 15: A. TADM curves of milk gels obtained by fermenting milk with single Streptococcus thermophilus strains in a 96-well micro-titer plate. B. Selected samples from (A), which resulted in milk gels with high to medium complex modulus (G*) values, related to gel firmness. A volume of 500 μl was aspirated. TADM curves are labelled with G* values of the corresponding samples.

### DESCRIPTION OF THE INVENTION

In a first aspect the present invention provides a method to determine at least one of shear stress, gel firmness and water-holding capacity of milk gels, said method comprising:
(i) providing one or more samples, each comprising a sample container and a sample, where at least one sample is said milk gel;
(ii) measurements of headspace pressure during aspiration and/or dispense of the samples of step (i) to obtain pressure versus time data; and
(iii) determining the at least one of shear stress, gel firmness and water-holding capacity of said milk gel from the pressure versus selected time data of step (ii);
wherein said shear stress is determined in step (iii) by correlating the pressure versus time data with shear stress; said gel firmness is determined in step (iii) by correlating the minimum pressure on the aspiration curve obtained from a non-mixed or centrifuged sample with complex modulus; and said water-holding capacity of the milk gels are determined in step (iii) by correlating the bend-time-point on the aspiration curve from a non-mixed or centrifuged sample with water-holding capacity.

The term “milk” is intended to mean any raw and/or processed milk material that can be subjected to fermentation according to the method of the invention. Thus, useful milk substrates include, but are not limited to, solutions/suspensions of any milk or milk-like products, such as whole or low fat milk, skim milk, buttermilk, reconstituted milk powder, condensed milk, dried milk, whey, whey permeate, lactose, whey protein concentrate, or cream. The milk substrate may originate from any mammal (such as a cow, a goat, or a pig) or reconstituted milk powder. Milk also includes liquids from plant material with a similar appearance which can be fermented by lactic acid bacteria to provide a milk gel. In a further embodiment the raw material is selected from low to high fat milk and from low to high protein milk. In a further embodiment the raw material is milk secreted from the mammary glands of a female mammal

The term “milk gel” as used herein is intended to mean any acidified milk gel, with high or low texture, and mixtures thereof, acidified by an acidifying agent such as microorganism, having at least one microorganism (such as a bacteria, a yeast, or a mold) carrying out fermentation in the milk, such as a dairy product, such as a fermented milk product (e.g. yoghurt, buttermilk, or sour cream), a cheese (e.g. fresh cheese).

As used herein the term “acidifying agent” is intended to mean any agent in solid, fluid or liquid form, which upon administration to a (micro-) container is able to lower the pH in the container compared to the pH in the container before adding the acidifying agent, such as a microorganism or an acid. In a further embodiment the acidifying agent is selected from a microorganism selected from bacteria, such as lactic acid bacteria.

In one embodiment the method of the present invention is for determination of shear stress, gel firmness and water-holding capacity.

In a further embodiment at least some of the samples are aspirated and dispensed for more than one aspiration and/or dispense cycle.

In a further embodiment said milk gels comprise more than one phase, such as a liquid phase and a soft solid phase.

In a further embodiment the samples are mixed after the first aspiration and/or dispense cycle and before the second aspiration and/or dispense cycle, e.g. by repeated aspiration and/or dispense or by a mixing device introduced into the sample container.

In a further embodiment, for at least one sample substantially the full sample volume is aspirated and/or dispensed in step (ii).

In a further embodiment the volume of each of said samples is less than 1 ml, less than 0.75 ml, about 0.5 ml or about 0.35 ml.

In a further embodiment at least some of the samples are aspirated and dispensed for more than one aspiration and/or dispense cycle.

In a further embodiment the milk gels are fermented milk products. In a further embodiment the milk gels are yoghurts, buttermilks, sour cream or fresh cheese. In a further embodiment the milk gels are yoghurts.

As shown in Examples 1 and 4, shear stress of the milk gels are inversely proportional with the headspace pressures at a selected time point on the aspiration curve and directly proportional with the headspace pressures at a selected time point on the dispense curve. As a result, shear stress can be determined by selection of a time point on the aspiration or dispense curve (depending on aspiration/dispense speed and volume) and correlation of the headspace pressure at this time point with shear stress using a model prepared by rheometer measurements.

Thus, in a further embodiment said shear stress of the milk gels are determined in step (iii) by correlating headspace pressure measured at a selected time point with shear stress.

As shown, in Example 3, there is also a very high predictive power of the linear regression model between shear stress and features from TADM pressure curves.

In a still further embodiment the pressure versus time data is obtained by aspirating.

In a further embodiment the pressure versus time data is obtained by dispensing.

In a still further embodiment the pressure versus time data is obtained by both aspirating and dispensing.

In a further embodiment the device used for step (ii) is an automated pipetting station equipped with a pressure sensor inside an air displacement barrel of each pipette. Typically, the device is a Hamilton Robotics MicroLab Star.

In a further embodiment the rheological properties determined are selected from one or more of Complex Modulus and Shear stress. Typically, the rheological properties determined are selected from Complex Modulus and Shear stress.

For high-throughput measurement of texturing properties of milk gels, milk is typically fermented directly in a micro container, typically a 96- or a 384-well micro-titer plate, and stored at 4° C. for 1 to 30 days at constant temperature, e.g. 4° C. Long storage times of about a month would result in spontaneous whey separation for some samples, which can be measured as water-holding capacity of milk gels by the method of the present invention. Other samples would show whey separation already during the milk fermentation. Alternatively, to avoid long storage times before the analysis of water-holding capacity is possible, an external force can be applied to the samples in micro-titer plates such as centrifugation to accelerate whey separation. Soft centrifugation (e.g. 500×g for 5-10 min) would typically provoke whey separation, but the centrifugation conditions might be different, depending on milk gel properties and milk content. For instance, a network of milk gels made of milk with lower amounts of fat and protein would be weaker than when milk gels are made of milk with higher amounts of fat and protein. Stabilizers such as pectin added to the milk would stabilize the milk gel network, and it would thus require a harder centrifugation (higher acceleration, longer times) to increase wheying-off. Too hard centrifugation would result in such a big damage of a gel network that a similar whey phase length for all the milk gel samples would be obtained, irrespectively of the strains used to ferment the milk.

In a further embodiment the sample container is one well in a multi-well plate.

In one embodiment the aspiration and/or dispense of the samples in step (ii) is from a pipette. In a further embodiment the tip of said pipette aspires the sample just below the surface of the sample such that the aspiration rate matches the speed at which the pipette tip is moved vertically into the sample.

In another embodiment at least some of the samples are centrifuged prior to said measurements for at least one aspiration and/or dispense cycle of step (ii).

In yet another embodiment the samples are subjected to a first aspiration and/or dispense cycle in step (ii) without having been centrifuged, centrifuging said samples and subsequently subjecting them to a further aspiration and/or dispense cycle in step (ii).

In yet another embodiment at least one sample is a standard.

In yet another embodiment the aspiration and/or dispense cycle or cycles of step (ii) excluding any centrifugation is completed for each sample within 60 seconds, within 30 seconds or within 5 seconds.

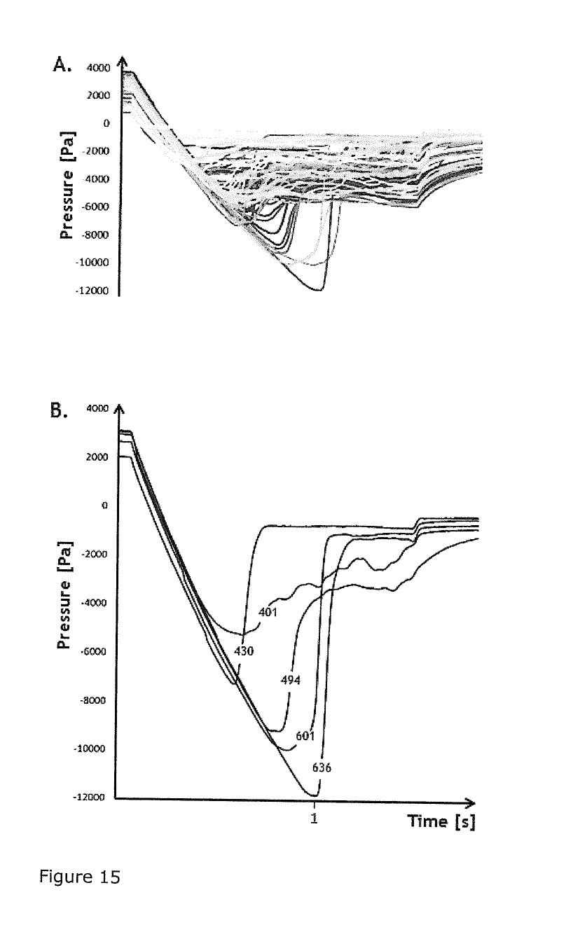

If water-holding capacity is determined for the samples that have not been stored for more than one day at 4° C., before the spontaneous whey separation has taken place, the method consists of the following steps: (i) Micro-titer plate containing fermented milk is centrifuged. As a result of centrifugation, two phases are formed: watery (whey) and viscous/solid; (ii) A 96-well robot head with conductive pipette tips enters the vials (96 samples simultaneously) and searches for the sample surface, using liquid level detection. If non-conductive pipette tips are used, it is necessary to define the distance from the bottom of the vials where the 96-well head would start to aspirate the samples; (iii) As soon as the tips sense the sample surface, they start aspirating the sample while moving slowly down into the vial, first into the whey phase and then into the viscous/solid phase. TADM is recorded simultaneously; (iv) Time point, at which TADM curves bend, indicating a shift from the whey to the solid phase, is recorded, and subsequently correlated with the whey phase length. Shorter times indicate a smaller whey phase lengths and thus a higher water-holding capacity (FIG. 15).

To determine water-holding capacity, shear stress and gel firmness of milk gel samples, typically in a 96-well micro-titer plate after a) storage at 4° C. or b) centrifugation, the plate is subjected to one or several subsequent aspiration and/or dispense steps, while TADM is recorded. During the first aspiration cycle, TADM pressure curves are presented in plots with pressure on the vertical axis (Pa) and time on the horizontal axis (milliseconds).The samples with V-like curves typically possess high gel firmness (FIG. 15). The lowest pressure observed for each sample with a V-shape like curve is correlated with gel firmness for milk gels with medium to high gel firmness. For samples with low gel firmness but relatively high shear stress, the correlation is not significant, as these pressure curves loose the V-shape, which makes gel firmness measurements difficult. However, as it is possible to spot samples with high to medium gel firmness with this method, it is a good tool for screening for strains with high gel firmness.

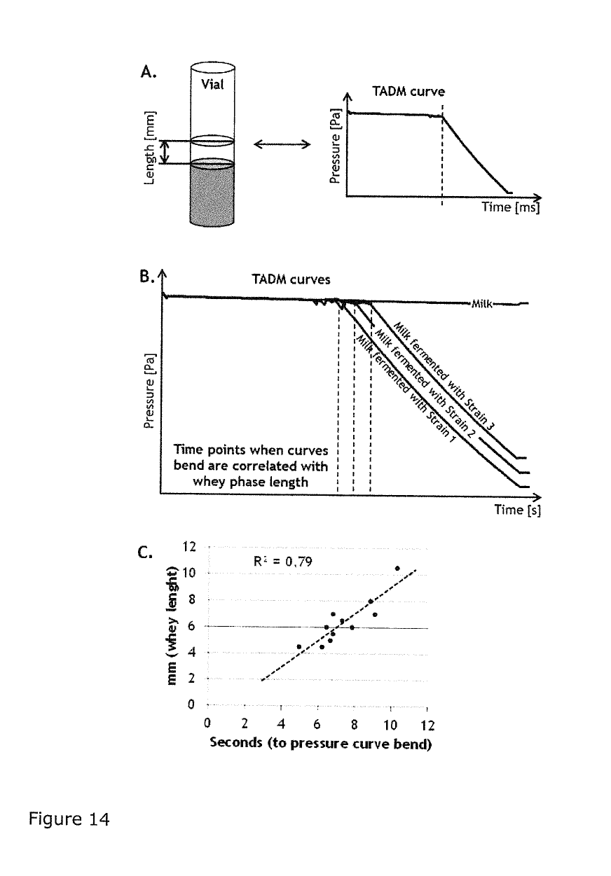

Water-holding capacity is determined in non-mixed samples during the first round of aspiration, when two phases (watery and solid) are present, by moving pipette tips slowly into the sample while aspirating, and recording the time point when the TADM pressure curve bends, indicating start of the solid phase (see FIG. 14).

The fermented milk gel may contain one microorganism, or several different microorganisms.

The fermented milk gel may be a mixture of two or more milk gels, or further additives may be mixed with the milk, such as stabilizers, gel forming agents, protein, e.g. skim milk powder, starch, carbohydrates.

In further embodiments, when aspirating and/or dispensing a sample from the milk gel, this is typically done within a short time interval, e.g. with a 96 channel pipetting head, 96 samples can be examined in a few seconds. Standard rheological measurements require 20 min per sample, so it requires 32 h for 96 samples.

Hence, when applying the method of the present invention for determining the rheological characteristics, a strongly increased number of samples can be analyzed, and thus an equivalent high number of milk compositions and microorganisms for producing milk gels can be analyzed, ranked and selected.

In still further embodiments, when aspirating and/or dispensing a sample from the milk gel, such sample is typically selected from a volume of from 100 microliter (μl) to 10 milliliter (ml). Typically, when the milk gel is a yoghurt, the sample aspirated and/or dispensed is selected from a volume of from 100 μl to 1 ml.

When aspirating and/or dispensing a sample from the milk gel, this is done within a certain temperature interval and at a certain pressure, which may vary depending on the milk gel to be tested. Milk gels are usually stored at low temperatures such as 4-13° C., although pressure measurements by TADM are typically done at room temperature, e.g. 20° C.

If the milk gel is yoghurt, the sample is aspirated and/or dispensed from the liquid within a temperature interval of from 4° C. to 30° C., preferably within a temperature interval of from 4° C. to 18° C., at ambient atmospheric pressure of about 1 atm.

Since the shear stress of milk gels are inversely proportional with the pressures versus time data on the aspiration curve and directly proportional with pressures versus time data on the dispense curve (shear stress of the milk gels are inversely proportional with the headspace pressures at a selected time point (depending on aspiration speed and volume) on the aspiration curve and directly proportional with the headspace pressures at a selected time point (depending on dispense speed and volume) on the dispense curve), the gel firmness of the milk gels are inversely proportional with the minimum pressures on the aspiration curve obtained from a non-mixed or centrifuged sample, and the water-holding capacity of the milk gels are inversely proportional with the bend-time-points on the aspiration curve from a non-mixed or centrifuged sample (see the Examples herein), shear stress, gel firmness and/or water-holding capacity of milk gel samples can be easily compared by comparing the relative pressure versus time data of the milk gel samples.

In a second aspect the present invention provides a method for the ranking of microorganisms according to their ability to contribute to shear stress, gel firmness or water-holding capacity of milk gels prepared in the presence of said microorganisms comprising the steps:
(i) providing two or more samples, each comprising a sample container and a sample, of a milk gel prepared in the presence of at least one microorganism;
(ii) measurements of headspace pressure during aspiration and/or dispense of the two or more samples of step (i) to obtain pressure versus time data; and
(iii) ranking the microorganisms according to the relative pressure versus time data of the milk gels prepared in the presence of the at least one microorganism,
   wherein shear stress is a) inversely proportional with pressure versus time data on the aspiration curve and b) directly proportional with pressure versus time data on the dispense curve; gel firmness is inversely proportional with the minimum pressure on the aspiration curve obtained from a non-mixed or centrifuged sample; and water-holding capacity is inversely proportional to the bend-time-point of the aspiration curve obtained from a non-mixed or centrifuged sample.

This method of the present invention may be very useful when it comes to determine or predict which microorganisms, such as bacteria, or mixtures of such microorganisms are suitable for producing a fermented milk, such as a yoghurt, which brings the desired shear stress, gel firmness and water-holding capacity and thereby the desired quality to the fermented milk product. In particular, microorganisms, such as bacteria, and mixtures thereof which leads to milk gels with undesired properties, such as relatively low or high viscosity, relatively low or high gel firmness or relatively low or high water-holding capacity, may be identified.

In a further embodiment the method for the ranking of microorganisms according to their ability to contribute to shear stress, gel firmness or water-holding capacity of milk gels prepared in the presence of said microorganisms further comprises in step (iv) selecting the at least one microorganism if it contributes to a relatively high shear stress, a relatively high gel firmness and/or a relatively high water-holding capacity of the milk gel prepared in the presence of the at least one microorganism.

In another embodiment the method for the ranking of microorganisms according to their ability to contribute to shear stress, gel firmness or water-holding capacity of milk gels prepared in the presence of said microorganisms further comprises in step (iv) selecting the at least one microorganism if it contributes to a relatively low shear stress, a relatively low gel firmness and/or a relatively low water-holding capacity of the milk gel prepared in the presence of the at least one microorganism.

In a further embodiment at least some of the samples are aspirated and/or dispensed for more than one aspiration and/or dispense cycle.

In a further embodiment said milk gels comprise more than one phase, such as a liquid phase and a soft solid phase.

In a further embodiment the samples are mixed after the first aspiration and/or dispense cycle and before the second aspiration and/or dispense cycle, e.g. by repeated aspiration and/or dispense or by a mixing device introduced into the sample container.

In a further embodiment, for at least one sample substantially the full sample volume is aspirated and/or dispensed in step (ii).

In a further embodiment the volume of each of said samples is less than 1 ml, less than 0.75 ml, about 0.5 ml or about 0.35 ml.

In a further embodiment at least some of the samples are aspirated and/or dispensed for more than one aspiration and/or dispense cycle.

In a further embodiment the milk gels are fermented milk products. In a further embodiment the milk gels are yoghurts, buttermilks, sour cream or fresh cheese. In a further embodiment the milk gels are yoghurts.

In a still further embodiment the pressure versus time data is obtained by aspirating.

In a further embodiment the pressure versus time data is obtained by dispensing.

In a still further embodiment the pressure versus time data is obtained by both aspirating and dispensing.

In a further embodiment the device used for step (ii) is an automated pipetting station equipped with a pressure sensor inside an air displacement barrel of each pipette. Typically, the device is a Hamilton Robotics MicroLab Star.

In a further embodiment the sample container is one well in a multi-well plate.

In one embodiment the aspiration and/or dispense of the samples in step (ii) is from a pipette.

In a further embodiment the tip of said pipette aspires the sample just below the surface of the sample such that the aspiration rate matches the speed at which the pipette tip is moved vertically into the sample.

In another embodiment at least some of the samples are centrifuged prior to said measurements for at least one aspiration and/or dispense cycle of step (ii).

In yet another embodiment the samples are subjected to a first aspiration and/or dispense cycle in step (ii) without having been centrifuged, centrifuging said samples and subsequently subjecting them to a further aspiration and/or dispense cycle in step (ii).

In yet another embodiment at least one sample is a standard.

In yet another embodiment the aspiration and/or dispense cycle or cycles of step (ii), excluding any centrifugation, is completed for each sample within 60 seconds, within 30 seconds or within 5 seconds.

The fermented milk gel may contain one microorganism, or several different microorganisms.

The fermented milk gel may be a mixture of two or more milk gels, or further additives may be mixed with the milk, such as stabilizers, gel forming agents, protein, e.g. skim milk powder, starch, carbohydrates.

In further embodiments, when aspirating and/or dispensing a sample from the milk gel, this is typically done within a short time interval, e.g. with a 96 channel pipetting head, 96 samples can be examined in a few seconds. Standard rheological measurements require 20 min per sample, so it requires 32 h for 96 samples.

In still further embodiments, when aspirating and/or dispensing a sample from the milk gel, such sample is typically selected from a volume of from 100 microliter (μl) to 10 milliliter (ml). Typically, when the milk gel is a yoghurt, the sample aspirated and/or dispensed is selected from a volume of from 100 μl to 1 ml.

When aspirating and/or dispensing a sample from the milk gel, this is done within a certain temperature interval and at a certain pressure, which may vary depending on the milk gel to be tested. Milk gels are usually stored at low temperatures such as 4-13° C., although pressure measurements by TADM are typically done at room temperature, e.g. 20° C.

If the milk gel is yoghurt, the sample is aspirated and/or dispensed from the liquid within a temperature interval of from 4° C. to 30° C., preferably within a temperature interval of from 4° C. to 18° C., at ambient atmospheric pressure of about 1 atm.

Furthermore, the present invention is very useful when it comes to screening for microorganisms, such as bacteria, or mixtures of such microorganisms, which are suitable for obtaining specific rheological properties of the milk gel.

Thus, the present method of determining shear stress may also be seen as a method of determining quality of a milk gel, by determining shear stress as described herein.

Milk gels may be measured in sample containers with 1, 2 or up to 384 containers with individual milk gel samples and possibly even higher well densities per area. In a further embodiment the high throughput screening is done in a micro container such as a micro-titer plate having at least 1, 8, such as at least 24, such as at least 48, such as at least 96, such as at least 384 wells. Typically, the high throughput screening is done in a micro-titer plate having 96 wells.

The microorganism used in the above method of monitoring is typically selected from lactic acid bacteria, such as Lactococcus, Lactobacillus, Streptococcus, Leuconostoc, Pediococcus, Bifidobacterium, Bacillus, Oenococcus, yeast, such as Saccharomyces, Pichia, Kluyveromyces, Candida and Geotrichum, molds such as Aspergillus and Penicillium.

The term “TADM” as used herein is intended to mean total aspiration and dispense monitoring, i.e. measurement of pressure during aspiration and dispense of a sample.

The term “additive” as used herein is intended to mean any suitable additive which is to be tested for impacting rheological properties and water-holding capacity of the milk gel. Such additives may be one additive or a mixture of two or more additives, to be mixed with the milk, such as without limitation stabilizers, gel forming agents, protein e.g. skim milk powder, starch, carbohydrates and hydrocolloids.

All of the embodiments described above in relation to the method of determining rheological properties, e.g. shear stress, gel firmness and water-holding capacity of a milk gel also apply to this method of monitoring changes in shear stress of a milk gel.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

Recitation of ranges of values herein are merely intended to serve as a short method of referring individually to each separate value falling within the range, unless other-wise indicated herein, and each separate value is incorporated into the specification as if it were individually recited herein.

Unless otherwise stated, all exact values provided herein are representative of corresponding approximate values (e.g. all exact exemplary values provided with respect to a particular factor or measurement can be considered to also provide a corresponding approximate measurement, modified by “about”, where appropriate).

All methods described herein can be performed in any suitable order unless other-wise indicated herein or otherwise clearly contradicted by context.

The terms “a” and “an” and “the” and similar referents as used in the context of de-scribing the invention are to be construed to cover both the singular and the plural, unless otherwise indicated herein or clearly contradicted by context. Thus, “a” and “an” and “the” may mean at least one, or one or more.

The use of any and all examples, or exemplary language (e.g. “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g. a composition described herein as comprising a particular element should be understood as also describing a composition consisting of that element, unless otherwise stated or clearly contradicted by context).

This invention includes all modifications and equivalents of the subject matter re-cited in the aspects or claims presented herein to the maximum extent permitted by applicable law.

The features disclosed in the foregoing description may, both separately and in any combination thereof, be material for realizing the invention in diverse forms thereof.

### EXAMPLES

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Equipment Used for Pipetting

A liquid handling station, Hamilton Robotics MicroLab Star, equipped with TADM tool was used in the following experiments.

The Hamilton Robotics MicroLab Star has 4, 8, 12, or 16 pipetting channels, as well as a 96-well head, built upon air-displacement technology, which is analogous to a hand held electronic pipette. Each channel can aspirate up to a volume of 1 ml, and for some pipetting channels, up to 5 ml. To aspirate within different volume ranges the 96-well head can accommodate a range of tips with volume from 10, 50, 300 to 1000 μl.

The liquid handler has a pressure sensor located in the headspace of each pipetting channel. Pressure data from each sensor is collected by suitable software running on a computer, for example, by the “Total Aspiration Dispense Monitoring” (TADM) software of the Hamilton Robotics MicroLab Star liquid handler (Hamilton Robotics).

### Example 1

### Commercial Yoghurt Cultures

Fermentation of Milk

Frozen concentrate of the yoghurt cultures were used to inoculate skim milk with 2% skim milk powder added. The milk was heated at 90° C. for 20 minutes prior to inoculation with 0.02% frozen concentrate. The incubation took place at 43° C. until pH reached 4.55, at which time the coagulation of the milk had taken place. Subsequently, a post treatment at 2 bar was applied to the yoghurt by a post treatment unit (PTU). The fermented milk was then cooled to 5° C.

Rheology Measurements in a Fermented Milk

Rheology: The day after incubation, the fermented milk was brought to 13° C. and stirred gently by means of a stick fitted with a perforated disc until homogeneity of the sample. The rheological properties of the sample were assessed on a rheometer (Anton Paar Physica Rheometer with ASC, Automatic Sample Changer, Anton Paar® GmbH, Austria). Settings were as follows:

Wait time (to rebuild to somewhat original structure)
5 minutes without oscillation or rotation

$$Oscillation ( to measure {G}^{′} and {G}^{″} for calculation of {G}^{*} ) = 0.3 \% , frequency ( f ) = [ 0.5 …8 ] Hz$$

6 measuring points over 60 s (one every 10 s)

$$Rotation ( to measure shear stress at 300 \; 1 \text{/} s ) = [ 0.3 \text{-} 300 ] 1 \text{/} s and = [ 275 \text{-} 0.3 ] 1 \text{/} s$$

21 measuring point over 210 s (on every 10 s) going up to 300 l/s.
and
21 measuring points over 210 s (one every 10 s) going down to 0.3 l/s

For further analysis the shear stress at 300 l/s was chosen.

Samples were also evaluated by a trained sensory panel, where gel firmness, mouth thickness, and ropiness was evaluated as previously described, visually by slowly placing a spoonful of yoghurt on an untouched yoghurt surface and evaluate how long it keeps the structure before flowing out—the longer the time the higher the Gel firmness.

Pipetting: Fermented milks prepared with cultures differing in textural properties were stirred uniformly to obtain homogeneity before transferring sample to test tubes. There was only one aspiration and dispensing pr. tube as the structure of the yoghurt will change when the tip enters. A volume of 500 μl was aspirated with a rate of 20 μl/sec and dispensed with 50 μl/sec and the pressure change recorded in real time.

Results and Conclusions

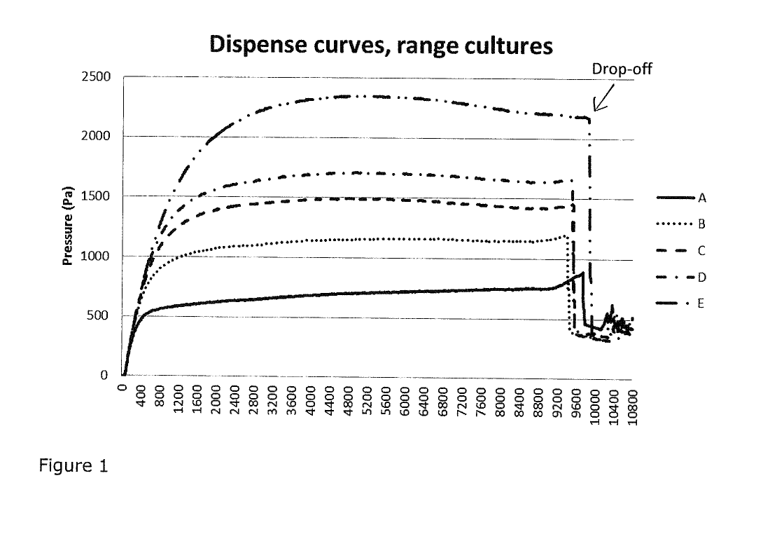

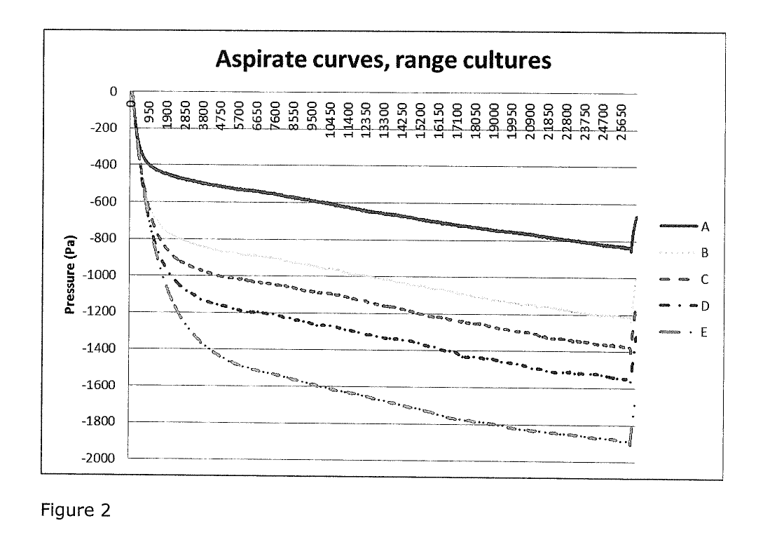

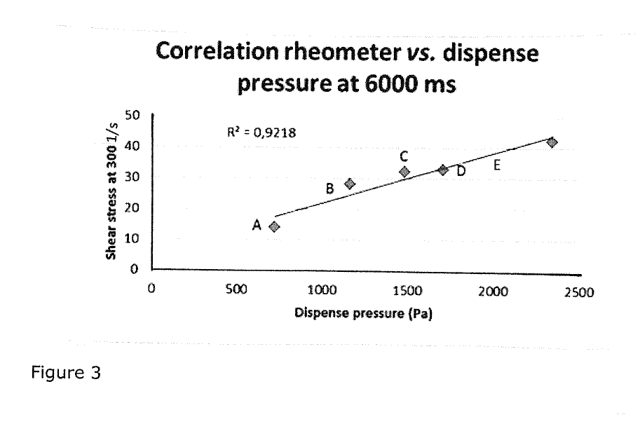

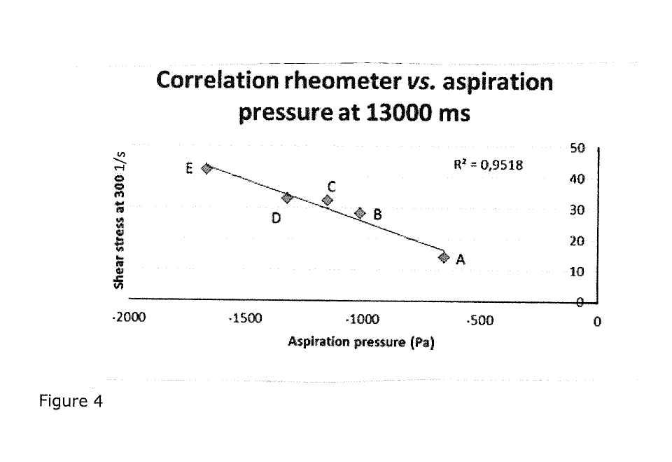

Fermented milks incubated with concentrates of yoghurt cultures that are currently used in the dairy industry and have different shear stress could be clearly differentiated by both the conventional rheometer test and by the aspiration and/or dispensing pressure monitored under pipetting (see FIGS. 1 and 2). Furthermore the correlation between the shear stress (Pa) at 300 s⁻¹, measured on the rheometer and dispensing pressure (Pa) showed a correlation coefficient of 0.92 (see FIG. 3). The correlation between the shear stress (Pa) at 300 s⁻¹, measured on the rheometer and aspiration pressure at 13 s showed a correlation coefficient of 0.95 (see FIG. 4).

### Example 2

### Single Strains of Lactobacilli

Fermentation of Milk

Two strains of Lactobacilli, differing in shear stress, were grown overnight in MRS broth at 37° C. The strains were inoculated by 1% v/v, in duplicate, in reconstituted milk with a dry matter content of 9.5%, which had been heat treated to 99° C. for 15 minutes in a batch process. The incubation took place at 37° C. until pH reached 4.55, at which time the coagulation of the milk had taken place. The fermented milk was stirred gently to homogeneity, cooled to 5° C. and the following day transferred to beakers.

Determination of Texture in a Fermented Milk

Pipetting: Fermented milks prepared with cultures differing in textural properties were stirred uniformly to obtain homogeneity before transferring sample to test tubes. There was only one aspiration and dispensing pr. tube. A volume of 500 μl was aspirated with a rate of 20 μl/sec and dispensed with a rate of 50 μl/sec and the pressure change recorded in real time.

Results and Conclusions

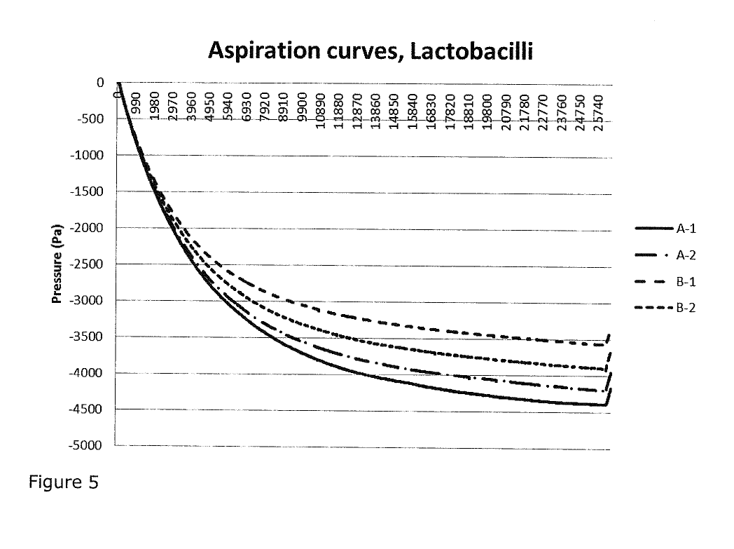

Fermented milks incubated with single strains of Lactobacilli having different rheological properties could be clearly differentiated by the aspiration pressure monitored during pipetting (see FIG. 5).

### Example 3

### Analysis of Texture in a Low Fat Yoghurt

Low Fat Yoghurts

Frozen concentrate of yoghurt cultures were used to inoculate skim milk (0.1% fat) with 2% skim milk powder added. The milk was heated at 90° C. for 20 minutes prior to inoculation with 0.02% frozen concentrate. The incubation took place at 43° C. until pH reached 4.55, at which time the coagulation of the milk had taken place. Subsequently, a post treatment at 2 bar was applied to the yoghurt by a post treatment unit (PTU). The fermented milk was then cooled to 5° C.

Rheology Measurements in Low Fat Yoghurt

Rheology: The day after incubation, the yoghurt was brought to 13° C. and stirred gently by means of a stick fitted with a bored disc until homogeneity of the sample. The rheological properties of the sample were assessed on a rheometer (Anton Paar Physica Rheometer with ASC, Automatic Sample Changer). Settings were as follows:

Wait time (to rebuild to somewhat original structure)
5 minutes without oscillation or rotation

$$Oscillation ( to measure {G}^{′} and {G}^{″} for calculation of {G}^{*} ) = 0.3 \% , frequency ( f ) = [ 0.5 …8 ] Hz$$

6 measuring points over 60 s (one every 10 s)

$$Rotation ( to measure shear stress at 300 \; 1 \text{/} s ) = [ 0.3 \text{-} 300 ] 1 \text{/} s and = [ 275 \text{-} 0.3 ] 1 \text{/} s$$

21 measuring point over 210 s (on every 10 s) going up to 300 l/s
and
21 measuring points over 210 s (one every 10 s) going down to 0.3 l/s

For further analysis the shear stress at 300 l/s was chosen.

Pipetting: Low fat yoghurts prepared as described above were transferred to test tubes. There was only one aspiration and dispensing pr. tube as the structure of the yoghurt will change when the tip enters. A volume of 500 μl was aspirated (25 μl/sec) and dispensed (65 μl/sec) and the pressure change was recorded in real time.

Pressure Curves Feature Extraction:

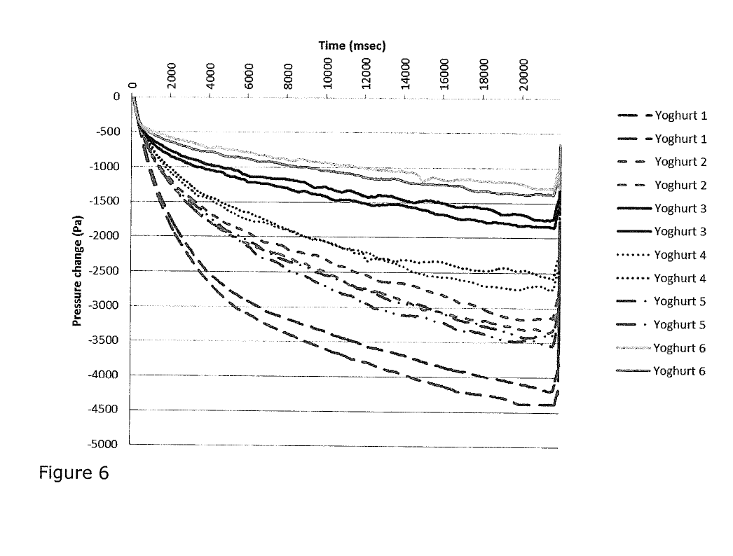

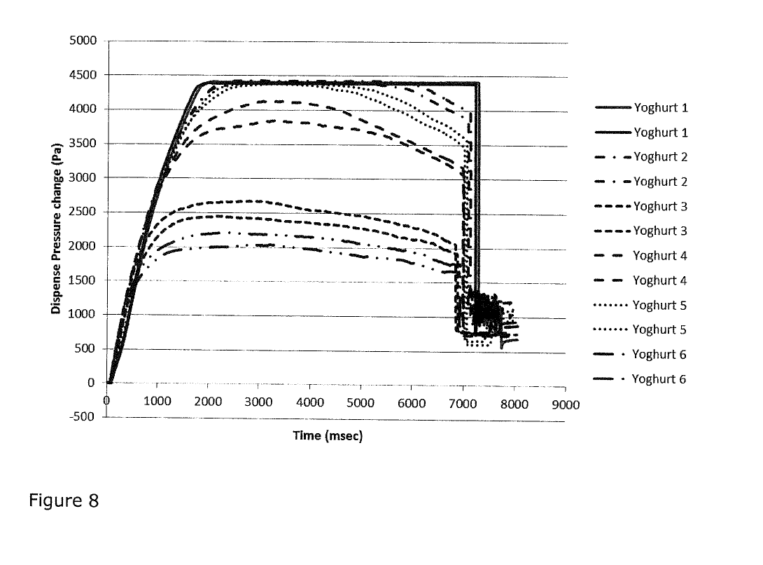

Aspiration and dispense curves obtained from 6 different low fat yoghurts (in duplicate) can be seen in FIGS. 6 and 8.

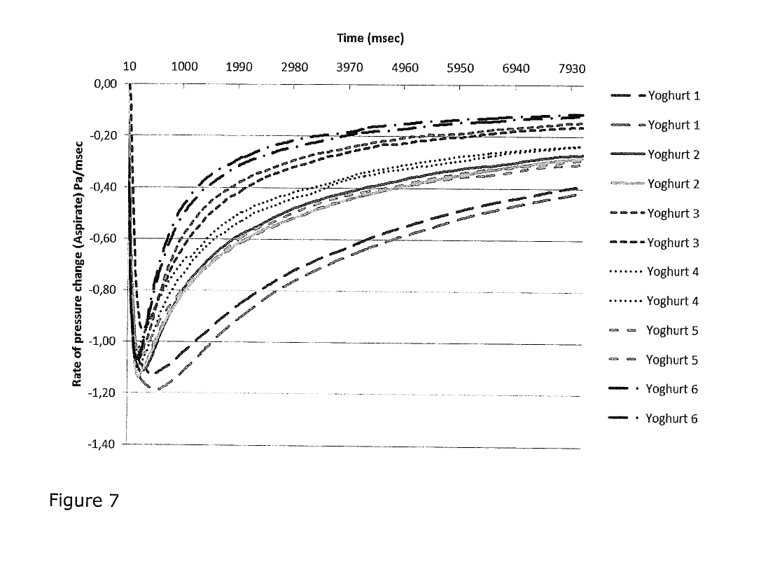

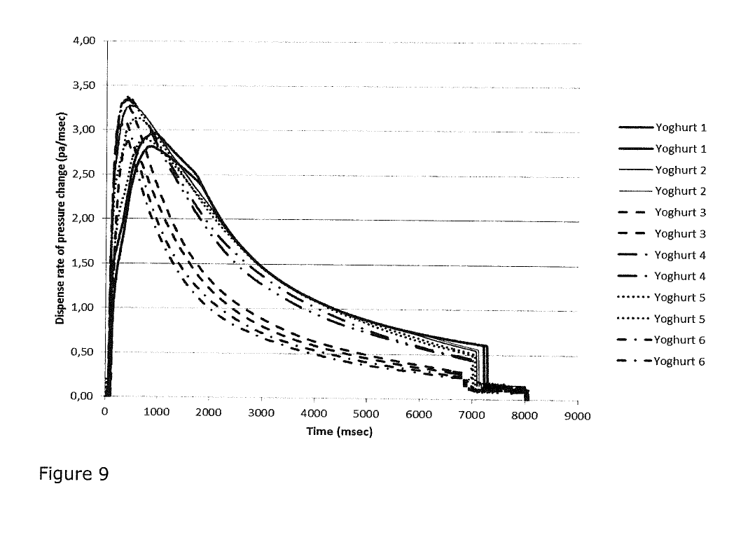

Instead of fixed arbitrary time points, a different approach for pressure curve features was chosen. Based on the pressure curves (FIG. 6) a calculation for the change of pressure rate is made (FIGS. 7 and 9). Then a max rate as well as the time of this to achieve are estimated as described here:

$$Aspirate rate = \frac{{Pa}_{t} - {Pa}_{0}}{t}$$

$$Max Aspirate rate = max ( Aspirate rate )$$

$$Aspirate {t}_{max} = t ( Max Aspirate rate )$$

$$Dispense rate = \frac{{Pa}_{t} - {Pa}_{0}}{t}$$

$$Max Dispense rate = max ( Dispense rate )$$

$$Dispense {t}_{max} = t ( Max Dispense rate )$$

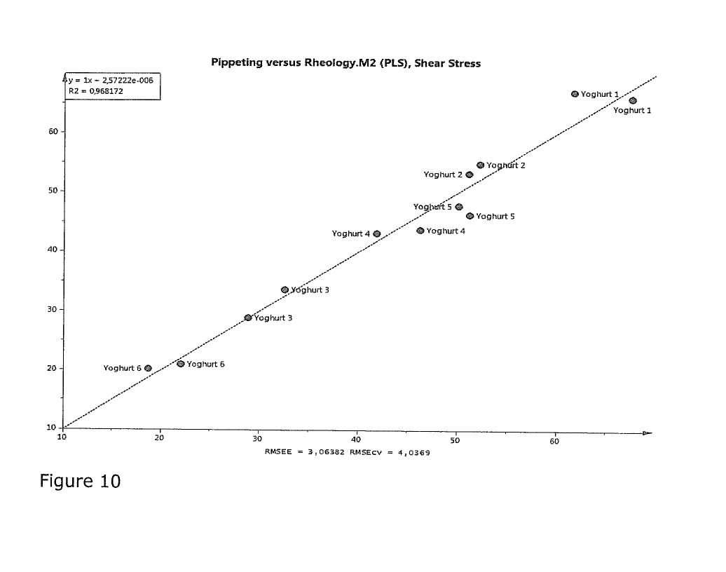

These descriptors (features) were used for partial least square analysis (see below and FIGS. 10-12). For the dispense curves specifically, a time offset at the end of dispensing occurs. This is called drop-off and it is used as a variable (in ms) (see FIGS. 1 and 8).

Results and Conclusions

Low fat stirred yoghurts incubated with concentrates of yoghurt cultures that are currently used in the dairy industry and have different rheological properties could be clearly differentiated by both the conventional rheometer test and by the aspiration and/or dispensing pressure monitored under pipetting (see FIGS. 6 and 8).

Additionally, features extracted from these curves were used to build Partial Least Squares (PLS) (Wold, Svante; Ruhe, Axel; Wold, Herman; Dunn, W. J. (1984). SIAM J. Sci. Stat. Comp. 5 (3): 735-743) models. These models examine linear regression relationships of these features to rheometer results of shear stress at 300 l/s and complex modulus at 1 Hz.

The models were built with SIMCA 13.0 by Umetrics.

The calculated features from the pressure curves were used as X variables (latent) while rheometer parameters were used as Y (reference) variables.

Linear regression models between X and Y variables were then established and linear regression coefficients were calculated for each of the X variables.

The resulting models are described for their ability to describe (R²) and predict (Q²) in the following table:

| | R² | Q² | RMSEE | RMSEcv |
|---|---|---|---|---|
| Shear Stress | 96.8% | 93.5% | 3.8% | 4.4% |
| G* | 92.4% | 78.6% | 7.9% | 11.5% |

The RMSEE (Root Mean Square Error of Estimation) indicates the fit of the observations to the model. The RMSEcv is an analogous measure, but estimated using the cross-validation (leave samples out) procedure and reflects the error for predictions. They represent the accuracy of the model.

The model error for “Shear Stress” is within reasonable limit (< or =10%), while “Complex modulus” (G*) is a less accurate in its prediction (11.5%). This inaccuracy is due to >20% deviations of the reference method (rheometer) especially at low levels of G* (<50)

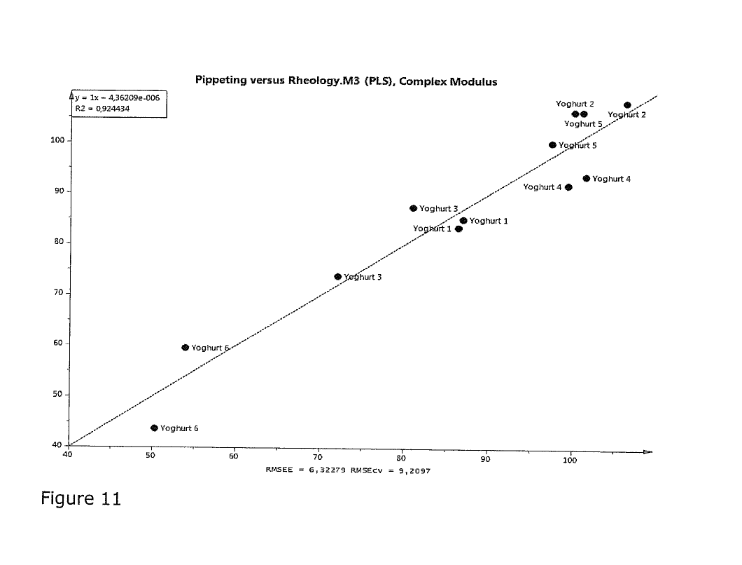

Furthermore, the predictive ability of shear stress at 300 l/s (Pa), measured on the rheometer by using the pipetting pressure features showed a correlation of 0.97 (see FIG. 10). The corresponding predictive model for Complex modulus by the pipetting features showed a correlation of 0.924 (see FIG. 11).

### Example 4

### High-Throughput Screening for Texturing LAB Strains

Fermentation of Milk

A 96-well micro-titer plate containing M17 broth with 1% lactose and 1% glucose was inoculated with different Lactococcus strains and incubated at 30° C. overnight in Galaxy R CO₂ incubator (RS Biotech). Another 96-well micro-titer plate containing M17 broth with 2% lactose was inoculated with different Streptococcus thermophilus strains and incubated at 37° C. overnight in Galaxy R CO₂ incubator. A 96-well micro-titer plate containing milk and pH indicator was inoculated with 1% overnight cultures from M17 broth with additives using Hamilton robot and incubated for 20 h at 30° C. for Lactococcus strains or for 18 h at 43° C. for Streptococcus strains. pH determination with pH indicator confirmed pH below 5.0 in most samples. There were 2 replicates of the plate. The plates containing acidified milk were stored at 4° C. for 1 day.

Selected Lactococcus and Streptococcus strains were used to inoculate milk in 200-mL volume, to be used for rheology analysis by a rheometer. The strains were inoculated by 1% v/v, in duplicate, in reconstituted milk with a dry matter content of 9.5%, which had been heat treated to 99° C. for 15 minutes in a batch process. The incubation took place at 30° C. for Lactococcus and at 43° C. for Streptococcus, until pH reached 4.55, at which time the coagulation of the milk had taken place. The fermented milk samples were stirred gently to homogeneity, cooled to 4° C. and were after 5 days at 4° C. transferred to beakers.

Analysis of Texture in a Fermented Milk

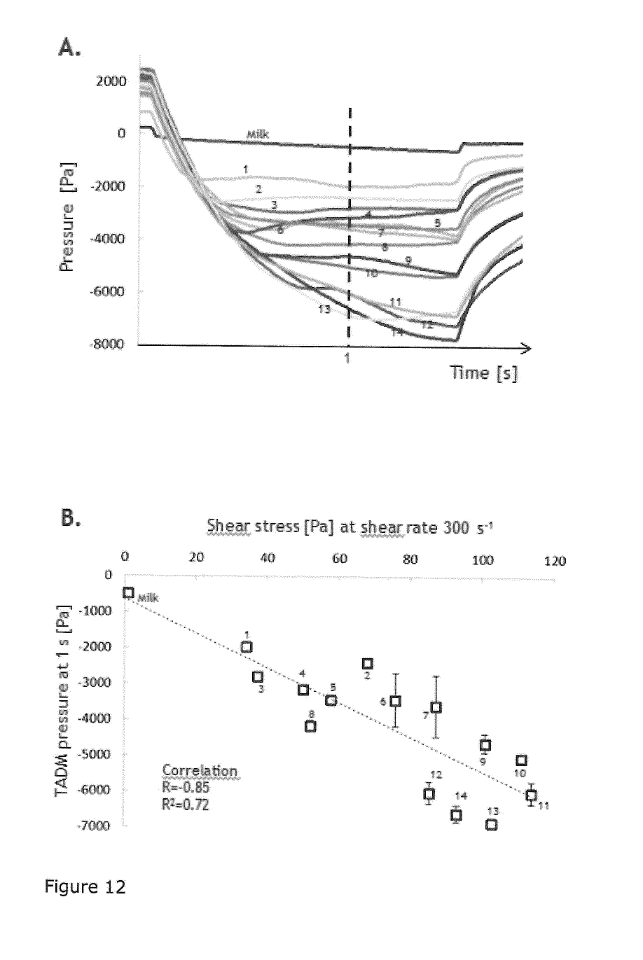

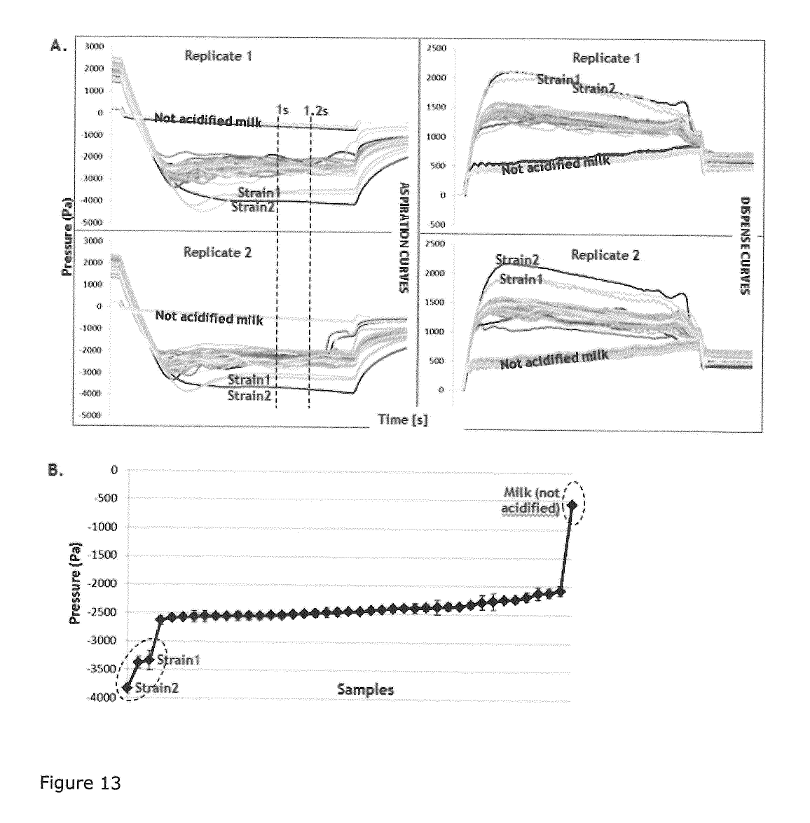

After milk acidification and storage at 4° C. for 1 day, the micro-titer plates were subjected to TADM pressure analysis by a Hamilton robot (FIG. 12A, 13A). A volume of 500 μl was aspirated (350 μl/s) using wide-bore tips. Aspiration pressure values at 1 s and 1.2 s for each sample from both replicates were averaged, sorted according to the pressure values (lowest to highest) and plotted (FIG. 13B).

A rheometer (Anton Paar Physica Rheometer with ASC, Automatic Sample Changer, Anton Paar® GmbH, Austria) was used to assess the rheological properties of the samples, using settings as in Example 1. Shear stress analysis at shear rate 300 s⁻¹ was chosen to correlate the rheological characteristics obtained from a rheometer and from the TADM analysis (FIG. 12B).

Results and Conclusions

By performing milk acidifications using different single strains in a micro-titer plate and analyzing the texturing properties of the resulting milk gels by TADM, it was possible to select strains resulting in high-viscous milk gels (FIG. 13) and milk gels with high gel firmness (FIG. 15).

On FIG. 13, both high-shear stress candidate strains, Strain 1 (present twice on the plate as high-shear stress control) and Strain 2 from (FIG. 13A), appear on the lower scale of (FIG. 13B), while milk samples and strains that did not acidify milk appear on the higher pressure scale (only one representative sample is shown). The majority of strains that have acidified the milk but did not result in high-viscous milk gel were situated between approximately −2000 and −2500 Pa under current measurement conditions.

TADM pressure analysis is thus a suitable tool for screening of microorganisms able to result in milk gels with desirable rheological properties.

### Example 5

### Determination of Water-Holding Capacity of Milk Gels

Water holding capacity of milk gels in e.g. set yogurt and/or fresh cheese is a desirable feature. To determine water-holding capacity of milk gel samples in a 96-well micro-titer plate, TADM pressure measurements are made on samples that have been centrifuged to induce wheying-off or alternatively have been stored cold for extended time, e.g. 30 days.

Fermentation of Milk

A 96-well micro-titer plate containing M17 broth with 2% lactose was inoculated with 3 different Streptococcus thermophilus strains, each strain was present as several replicates to assess the level of variation, and incubated at 37° C. overnight in Galaxy R CO₂ incubator (RS Biotech). A 96-well micro-titer plate containing milk and pH indicator was inoculated with 1% overnight cultures from M17 broth with 2% lactose using Hamilton robot and incubated for 18 h at 43° C.

Determination of Water-Holding Capacity of a Fermented Milk

The plate containing milk gel samples and milk as control was centrifuged at 500×g for 10 min at 4° C. Water-holding capacity was determined in non-mixed samples during the first round of aspiration, when two phases (whey and gel/solid) were present, by moving pipette tips slowly into the sample while aspirating, and recording the time point when the TADM pressure curve changed significantly, indicating a start of the solid phase (FIG. 14). A volume of 700 μl was aspirated (60 μl/s) using conductive pipette tips, to sense the start of the sample (and thus a start of aspiration).

Results and Conclusions

A correlation was found (R²=0.79) between whey length, as measured by a ruler, which represents syneresis (wheying-off or water-holding capacity), and the time point when TADM pressure curves change significantly (bend-time-point) (FIG. 14A and C).

Milk acidifications in micro-titer plates using different strains is a suitable tool for selecting strains for e.g. set yoghurt and fresh cheese, where strains resulting in milk gel with high water-holding capacity are desired. The extraction of the data can be done in an automated way from e.g. an Excel file, and based on the results, milk gel samples with desired properties and the corresponding cultures, additives, and culture conditions can be found.

### REFERENCES

Lee W J, Lucey J A (2010) Formation and physical properties of yogurt. Asian-Aust J Anim Sci 23: 1127-1136

Mezger T G (2006) The Rheology Handbook: For users of rotational and oscillatory rheometers, 2nd revised edition, Hannover Network.

WO2002073215

EP1614468

WO2011107472

EP2009449

U.S. Pat. No. 6,938,504

WO2008153387

1. A method for determining at least one of shear stress, gel firmness and water-holding capacity of a milk gel, comprising:
(i) providing a container containing a milk gel;
(ii) subjecting a sample of said milk gel to at least one aspiration/dispense cycle and measuring headspace pressure during aspiration and/or dispense of said sample to obtain pressure versus time data; and
(iii) determining at least one of shear stress, gel firmness and water-holding capacity of said milk gel from the pressure versus time data obtained in step (ii), wherein
said shear stress is determined by correlating the pressure versus time data with shear stress;
said gel firmness is determined by correlating the minimum pressure of the pressure versus time data measured during aspiration of a non-mixed or centrifuged sample of said milk gel with complex modulus; and
said water-holding capacity is determined in step (iii) by correlating the bend-time-point of the pressure versus time data measured during aspiration of a non-mixed or centrifuged sample of said milk gel with water-holding capacity.

2. The method according to claim 1, wherein shear stress is determined.

3. The method according to claim 1, wherein gel firmness is determined.

4. The method according to claim 1, wherein shear stress and gel firmness are both determined.

5. The method according to claim 1, wherein water-holding capacity is determined.

6. The method according to claim 1, wherein said milk gel is subjected to more than one aspiration/dispense cycle.

7. The method according to claim 1, wherein said milk comprises a liquid phase and a soft solid phase.

8. The method according to claim 6, wherein said milk gel is subjected to a first aspiration/dispense cycle to obtain a first set of pressure versus time data, then said milk gel is mixed before a second aspiration/dispense cycle, and then said milk gel is subjected to a second aspiration/dispense cycle to obtain a second set of pressure versus time data.

9. The method according to claim 1, wherein said milk gel is a fermented milk product.

10. The method according to claim 1, wherein said milk gel is selected from the group consisting of yoghurts, buttermilks, sour cream and fresh cheese.

11. The method according to claim 1, wherein said milk gel is centrifuged prior to at least one aspiration/dispense cycle of step (ii).

12. The method according to claim 1, wherein said milk gel is subjected to a first aspiration/dispense cycle of step (ii) without having been centrifuged, then is subjected to centrifuging, and then is subjected to a further aspiration/dispense cycle of step (ii).

13. The method according to claim 1, wherein the apparatus used for step (ii) is a Hamilton Robotics MicroLab Star.

14. A method for ranking microorganisms according to their ability to contribute to shear stress, gel firmness and/or water-holding capacity of milk gels prepared in the presence of said microorganisms, comprising:
(i) providing two or more containers, each containing a milk gel prepared in the presence of at least one microorganism;
(ii) subjecting a sample of each of said milk gels to an aspiration/dispense cycle and measuring headspace pressure during aspiration and/or dispense of the samples to obtain pressure versus time data for each milk gel; and
(iii) ranking the microorganisms according to the relative pressure versus time data of the milk gels prepared in their presence, wherein
shear stress is inversely proportional with pressure versus time data measured during aspiration and directly proportional with pressure versus time data measured during dispense;
gel firmness is inversely proportional with the minimum pressure of the pressure versus time data measured during aspiration of a non-mixed or centrifuged sample; and
water-holding capacity is inversely proportional with the bend-time-point of pressure versus time data measured during aspiration of a non-mixed or centrifuged sample.

15. The method according to claim 14, further comprising:
(iv) selecting the at least one microorganism if it contributes to a relatively high shear stress, a relatively high gel firmness and/or a relatively high water-holding capacity of the milk gel prepared in the presence of the at least one microorganism.

16. The method according to claim 1, wherein the measuring comprises total aspiration and dispense monitoring (TADM).

17. The method according to claim 1, wherein the container is a well of a microtiter plate.

18. The method according to claim 17 wherein the aspiration/dispense cycle is performed with a microtiter plate pipetting system.

19. The method according to claim 18, wherein the microtiter plate pipetting system comprises pipettes equipped with pressure sensors.

20. The method according to claim 1, wherein the method is conducted as a high-throughput method of multiple milk gels, each provided in a well of a microtiter plate equipped with a microtiter plate pipetting system comprising pipettes equipped with pressure sensors.
